(19) United States
(12) Patent Application Publication
(10) Pub. No.: US 2025/0069701 A1
(43) Pub. Date: Feb. 27, 2025

(54) Deep Learning Methods For Biosynthetic Gene Cluster Discovery

(71) Applicant: LifeMine Therapeutics, Inc., Cambridge, MA (US)

(72) Inventors: Michalis HADJITHOMAS, Cambridge, MA (US); Michael Qi DING, Cambridge, MA (US); Demetrius Michael DIMUCCI, Cambridge, MA (US); Nancy Ann ZHANG, Cambridge, MA (US); Iain James MCFADYEN, Cambridge, MA (US); Gregory L. VERDINE, Cambridge, MA (US)

(21) Appl. No.: 18/668,729

(22) Filed: May 20, 2024

Related U.S. Application Data
(63) Continuation of application No. PCT/US2022/080447, filed on Nov. 23, 2022.
(60) Provisional application No. 63/282,451, filed on Nov. 23, 2021.

(51) Int. Cl. G16B 40/00; G16B 30/10

(52) U.S. Cl. CPC G16B 40/00; G16B 30/10

(57) ABSTRACT

The present disclosure relates to computer-implemented methods and systems for identifying biosynthetic gene clusters (BGCs) that encode pathways for the production of secondary metabolites. Secondary metabolites that target genes or gene products that are homologous to, e.g., human genes or gene products may have utility as potential drug compounds.

### CROSS-REFERENCE TO RELATED APPLICATIONS

This application is a continuation of International Application No. PCT/US22/080447, which designated the United States and was filed on Nov. 23, 2022, published in English, which claims the priority benefit of U.S. Provisional Patent Application Ser. No. 63/282,451, filed Nov. 23, 2021, the contents of which are incorporated herein by reference in their entirety.

### FIELD

The present disclosure relates generally to methods and systems for identifying genes associated with biosynthetic gene clusters, and applications thereof, including identifying potential therapeutic targets and drug candidates.

### BACKGROUND

Microbes produce a wide variety of small molecule compounds, known as secondary metabolites or natural products, which have diverse chemical structures and functions. Some secondary metabolites allow microbes to survive adverse environments, while others serve as weapons of inter-and intra-species competition. See, e.g., Piel, J. Nat. Prod. Rep., 26:338-362, 2009. Many human medicines (including, e.g., antibacterial agents, antitumor agents, and insecticides) have been derived from secondary metabolites. See, e.g., Newman D. J. and Cragg G. M. J. Nat. Prod., 79:629-661, 2016.

Microbes synthesize secondary metabolites using enzyme proteins encoded by clusters of co-located genes called biosynthetic gene clusters (BGCs). Evidence is emerging that some microbial biosynthetic gene clusters contain genes that appear not to be involved in synthesis of the relevant biosynthetic products produced by the enzymes encoded by the clusters. In some cases, such non-biosynthetic genes have been described as “self-protective” because they encode proteins that apparently can render the host organism resistant to the relevant biosynthetic product. For example, in some cases, genes encoding transporters of the biosynthetic products, detoxification enzymes that act on the biosynthetic products, or resistant variants of proteins whose activities are targeted by the biosynthetic products, have been reported. See, for example, Cimermancic, et al., Cell 158:412, 2014; Keller, Nat. Chem. Biol. 11:671, 2015. In some cases, such genes may be referred to as “resistance genes”. Researchers have proposed that identification of such genes, and determination of their functions, could be useful in determining the role of the biosynthetic products synthesized by the enzymes of the clusters. See, for example, Ych, et al., ACS Chem. Biol. 11:2275, 2016; Tang, et al., ACS Chem. Biol. 10:2841, 2015; Regueira, et al., Appl, Environ. Microbiol. 77:3035, 2011; Kennedy, et al., Science 284:1368, 1999; Lowther, et al., Proc. Natl. Acad. Sci. USA 95:12153, 1998; Abe, et al., Mol. Genet. Genomics 268:130, 2002. United States Patent Application Publication No. 2020/0211673 A1 provides insights that certain genes present in biosynthetic gene clusters, or located in close proximity to the biosynthetic genes of the clusters (particularly in eukaryotic, e.g., fungal, biosynthetic gene clusters as contrasted with bacterial biosynthetic gene clusters) may represent homologs of human genes that are targets of therapeutic interest. Such genes are referred to as “embedded target genes” (“ETaGs”) or “non-embedded target genes” (NETaGs) depending on whether or not they are located within the cluster of biosynthetic genes.

Traditionally, secondary metabolites have been identified from microbial cultures and screened for therapeutic activities against human targets of interest. However, the vast majority of microbes are not culturable, and even BGCs in culturable microbes can remain transcriptionally silent under laboratory conditions. Recent developments in nucleic acid and protein sequencing technologies and bioinformatics pipelines have enabled rapid identification of a large number of BGCs from environmental microbes without having to culture the microbes and test the bioactivity of the BGCs. See, e.g., Palazzotto E. and Weber T. Curr. Opin. Microbiol., 45:109-116, 2018. However, it remains a challenge to precisely define the genomic boundaries of BGCs using pure computational methods. There are also no computational pipelines available to identify genes associated with (but not embedded within) biosynthetic gene clusters, or for predicting the function of secondary metabolites and predicting biosynthetic gene clusters that produce secondary metabolites having an activity of interest.

### SUMMARY

Disclosed herein are computer-implemented methods and systems for identifying genes associated with biosynthetic gene clusters (BGCs) that may be used for, e.g., identifying BGCs that encode for potential drug compounds. BGC identification is performed via the application of advanced machine learning techniques. Innovations for computational BGC discovery include: novel data representations, novel application of advanced model architectures, and novel ensemble learning models comprising separate computational models. In some instances, training data is generated from a proprietary dataset of BGCs with high-confidence boundaries. Genome encoded molecule (GEM) compound class and function prediction is performed via a transfer learning framework with novel feature sets.

Disclosed herein are computer-implemented methods for identifying biosynthetic gene clusters comprising: receiving a first representation of at least one first genome as input; processing the representation of the at least one first genome using a trained machine learning model configured to detect patterns of predicted protein domains encoded by genes belonging to a biosynthetic gene cluster (BGC); and outputting, based on detection of a pattern of predicted protein domains corresponding to a BGC in the first representation of the at least one first genome, a second representation of the at least one first genome that identifies a set of genes that belong to the BGC.

In some embodiments, the first representation of the at least one first genome comprises a nucleotide sequence for the at least one first genome. In some embodiments, the first representation of the at least one first genome comprises a vector representation of the at least one first genome, or an embedding thercof.

In some embodiments, the first representation of the at least one first genome comprises a sequence of CDD, Gene3D, PANTHER, Pfam, ProSitePatterns, ProSiteProfiles, SUPERFAMILY, SMART, TIGRFAM, SFLD, Hamap, Coils, PRINTS, PIRSR, AntiFam, MobiDBLite, or PIRSF representations, or any combination thereof, of protein domains encoded by genes within the at least one first genome. In some embodiments, the sequence of protein domain representations is generated by a process comprising retrieving a protein sequence for cach gene in the at least one first genome and identifying protein domains in the protein sequence by sequence alignment against a database of CDD, Gene3D, PANTHER, Pfam, ProSitePatterns, ProSiteProfiles, SUPERFAMILY, SMART, TIGRFAM, SFLD, Hamap, Coils, PRINTS, PIRSR, AntiFam, MobiDBLite, or PIRSF representations, respectively. In some embodiments, the first representation of the at least one first genome further comprises associated gene ontology (GO) terms, an identification of any resistance genes present, an identification of additional regulatory elements, or an identification of additional epigenetic elements.

In some embodiments, the computer-implemented method further comprises encoding each protein domain representation in the sequence of CDD, Gene3D, PANTHER, Pfam, ProSitePatterns, ProSiteProfiles, SUPERFAMILY, SMART, TIGRFAM, SFLD, Hamap, Coils, PRINTS, PIRSR, AntiFam, MobiDBLite, or PIRSF representations of protein domains as a vector representation of the at least one first genome using a representation learning system. In some embodiments, the representation learning system comprises a graphical learning model, a deep autoencoder, Pfam2vec, word2vec, GloVe, or fastText.

In some embodiments, the first representation of the at least one first genome comprises a sequence of annotations of genes within the at least one first genome based on a gene function and pathway mapping database. In some embodiments, the pathway mapping database is KEGG.

In some embodiments, the first representation of the at least one first genome comprises a sequence of annotations of genes within the at least one first genome based on a database comprising data for clusters of orthologous groups (COGs). In some embodiments, the database comprising data for clusters of orthologous groups (COGs) is EggNOG.

In some embodiments, the computer-implemented method further comprises encoding the sequence of gene annotations as a vector representation of the at least one first genome using a representation learning system. In some embodiments, the representation learning system comprises a graphical learning model, a deep autoencoder, Pfam2vec, word2vec, GloVe, or fastText.

In some embodiments, the trained machine learning model comprises a deep learning model. In some embodiments, the deep learning model comprises a supervised learning model or an unsupervised learning model. In some embodiments, the deep learning model comprises a convolutional neural network, a long short-term memory network, or a transformer model. In some embodiments, the deep learning model comprises a combination of components from a neural network, a convolutional neural network, a long short-term memory network, or a transformer neural network.

In some embodiments, the machine learning model is trained using a training data set comprising data for a plurality of training genomes. In some embodiments, the plurality of training genomes comprises a plurality of synthetic training genomes. In some embodiments, one or more synthetic training genomes of the plurality of synthetic training genomes each comprise a set of gene sequences from an actual BGC randomly inserted into a BGC negative genome. In some embodiments, one or more synthetic training genomes of the plurality of synthetic training genomes each comprise a set of gene sequences from a combination of actual positive BGC examples and synthetic negative BGC examples.

In some embodiments, the second representation of the at least one first genome comprises a vector representation, a graph representation, or a tensor representation of the at least one first genome.

In some embodiments, the computer-implemented method further comprises evaluating a gene identified as belonging to the BGC to determine if it is a resistance gene. In some embodiments, the resistance gene is an embedded target gene (ETaG) or a non-embedded target gene (NETaG). In some embodiments, the computer-implemented method further comprises performing an in vitro assay to test a secondary metabolite produced by the BGC in the at least first genome to which the resistance gene has been identified as belonging for activity against a resistance gene homolog, or protein encoded thereby, identified in a second genome that differs from the at least one first genome. In some embodiments, the computer-implemented method further comprises performing an in vivo assay to test a secondary metabolite produced by the BGC in the at least first genome to which the resistance gene has been identified as belonging for activity against a resistance gene homolog, or protein encoded thereby, identified in a second genome that differs from the at least one first genome. In some embodiments, the second genome comprises a mammalian genome, a human genome, an avian genome, a reptilian genome, an amphibian genome, a plant genome, a fungal genome, a bacterial genome, or a viral genome.

In some embodiments, the at least one first genome comprises a eukaryotic genome or a prokaryotic genome. In some embodiments, the at least one first genome is a eukaryotic genome, and the eukaryotic genome comprises a plant genome or a fungal genome. In some embodiments, the at least one first genome is a prokaryotic genome, and the prokaryotic genome is a bacterial genome.

In some embodiments, the first representation of the at least one first genome is input by a user of a system configured to perform the computer-implemented method.

Disclosed herein are computer-implemented methods comprising: receiving a sequence for at least one first genome as input; generating a first representation of the at least one first genome, wherein the first representation of the at least one first genome comprises a sequence of protein domain representations encoded by genes within the at least one first genome; and encoding cach protein domain representation in the sequence of protein domain representations as a vector representation of the at least one first genome using a representation learning system.

In some embodiments, the sequence of protein domain representations encoded by genes within the at least one first genome comprises a sequence of CDD, Gene3D, PANTHER, Pfam, ProSitePatterns, ProSiteProfiles, SUPERFAMILY, SMART, TIGRFAM, SFLD, Hamap, Coils, PRINTS, PIRSR, AntiFam, MobiDBLite, or PIRSF representations, or any combination thereof, of protein domains encoded by genes within the at least one first genome. In some embodiments, the sequence of protein domain representations is generated by a process comprising retrieving a protein sequence for each gene in the at least one first genome and identifying protein domains in the protein sequence by sequence alignment against a database of CDD, Gene3D, PANTHER, Pfam, ProSitePatterns, ProSiteProfiles, SUPERFAMILY, SMART, TIGRFAM, SFLD, Hamap, Coils, PRINTS, PIRSR, AntiFam, MobiDBLite, or PIRSF representations, respectively. In some embodiments, the first representation of the at least one first genome further comprises associated gene ontology (GO) terms, an identification of any resistance genes present, an identification of additional regulatory elements, or an identification of additional epigenetic elements. In some embodiments, the representation learning system comprises a graphical learning model, a deep autoencoder, Pfam2vec, word2vec, GloVe, or fastText. In some embodiments, the representation learning system is trained on a corpus of annotated genomes, each comprising a sequence of CDD, Gene3D, PANTHER, Pfam, ProSitePatterns, ProSiteProfiles, SUPERFAMILY, SMART, TIGRFAM, SFLD, Hamap, Coils, PRINTS, PIRSR, AntiFam, MobiDBLite, or PIRSF representations, or any combination thereof, of protein domains encoded by genes within a genome of the corpus.

Also disclosed herein are systems comprising: one or more processors; and a memory communicatively coupled to the one or more processors and configured to store instructions that, when executed by the one or more processors, cause the system to perform any of the methods described herein.

Also disclosed herein are non-transitory computer-readable storage media storing one or more programs, the one or more programs comprising instructions which, when executed by one or more processors of a system, cause the system to perform any of the methods described herein.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE FIGURES

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a process flowchart for predicting genes that belong to a biosynthetic gene cluster (BGC) in a first representation of a genome.

FIG. 2 provides a non-limiting example of a process flowchart for generating an embedded representation of a genome as an ordered list of vectors, each of which represents a specific protein domain, e.g., a Pfam domain, or annotation.

FIG. 3 provides a non-limiting schematic illustration of a computing device in accordance with one or more examples of the disclosure.

### DETAILED DESCRIPTION

Disclosed herein are computer-implemented methods and systems for identifying genes associated with biosynthetic gene clusters (BGCs) that may be used for, e.g., identifying BGCs that encode for potential drug compounds. BGC identification is performed via the application of advanced machine learning techniques. Innovations for computational BGC discovery include: novel data representations, novel application of advanced model architectures, and novel ensemble learning models comprising separate computational models. In some instances, training data is generated from a proprietary dataset of BGCs with high-confidence boundaries. Genome encoded molecule (GEM) compound class and function prediction is performed via a transfer learning framework with novel feature sets.

In some instances, for example, the disclosed methods (e.g., computer-implemented methods) may comprise: receiving a first representation of at least one first genome as input; processing the representation of the at least one first genome using a trained machine learning model configured to detect patterns of predicted protein domains encoded by genes belonging to a biosynthetic gene cluster (BGC); and outputting, based on detection of a pattern of predicted protein domains corresponding to a BGC in the first representation of the at least one first genome, a second representation of the at least one first genome that identifies a set of genes that belong to the BGC.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly indicates otherwise. Any reference to “or” herein is intended to encompass “and/or” unless otherwise stated, and encompasses any and all possible combinations of one or more of the associated listed items.

As used herein, the terms “includes, “including,” “comprises,” and/or “comprising” specify the presence of stated features, integers, steps, operations, elements, components, and/or units but do not preclude the presence or addition of one or more other features, integers, steps, operations, elements, components, units, and/or groups thereof.

As used herein, the term “about” a number refers to that number plus or minus 10% of that number. The term ‘about’ when used in the context of a range refers to that range minus 10% of its lowest value and plus 10% of its greatest value.

As used herein, a “secondary metabolite” refers to an organic small molecule compound produced by archaea, bacteria, fungi or plants, which is not directly involved in the normal growth, development, or reproduction of the host organism, but are required for interaction of the host organism with its environment. Secondary metabolites are also known as natural products or genetically encoded small molecules. The term “secondary metabolite” is used interchangeably herein with “biosynthetic product” when referring to the product of a biosynthetic gene cluster.

The terms “biosynthetic gene cluster” or “BGC” are used herein interchangeably to refer to a locally clustered group of one or more genes that together encode a biosynthetic pathway for the production of a secondary metabolite. Exemplary BGCs include, but are not limited to, biosynthetic gene clusters for the synthesis of non-ribosomal peptide synthetases (NRPS), polyketide synthases (PKS), terpenes, and bacteriocins. See, for example, Keller N, “Fungal secondary metabolism: regulation, function and drug discovery.” Nature Reviews Microbiology 17.3 (2019): 167-180 and Fischbach M. and Voigt C. A., PROKARYOTIC GENE CLUSTERS: A RICH TOOLBOX FOR SYNTHETIC BIOLOGY. In: Institute of Medicine (US) Forum on Microbial Threats. The Science and Applications of Synthetic and Systems Biology: Workshop Summary. Washington (DC): National Academies Press (US); 2011. A21. BGCs contain genes encoding signature biosynthetic proteins that are characteristic of each type of BGC. The longest biosynthetic gene in a BGC is referred to herein as the “core synthase gene” of a BGC. In addition to genes involved in the biosynthesis of a secondary metabolite, a BGC may also include other genes, e.g., genes that encode products that are not involved in the biosynthesis of a secondary metabolite, which are interspersed among the biosynthetic genes. These genes are referred to herein as being “associated” with the BGC if their products are functionally related to the secondary metabolite of the BGC. Some genes, e.g., genes not involved in the biosynthesis of a secondary metabolite produced by a BGC, are referred to herein as being “embedded” in the BGC if their products are functionally related to the secondary metabolite of the BGC and they are physically located in close proximity to the biosynthetic genes of the cluster. Some genes, e.g., genes not involved in the biosynthesis of a secondary metabolite produced by a BGC, are referred to herein as “non-embedded” if their products are functionally related to the secondary metabolite of a BGC but they are not physically located in close proximity to the biosynthetic genes of the BGC. An “anchor gene” refers to a biosynthetic gene or a gene that is not involved in the biosynthesis of a secondary metabolite produced by a BGC that is co-localized with a BGC and is known to be functionally related (i.e., associated) with the BGC.

The term “co-localize” refers to the presence of two or more genes in close spatial positions, such as no more than about 200 kb, no more than about 100 kb, no more than about 50 kb, no more than about 40 kb, no more than about 30 kb, no more than about 20 kb, no more than about 10 kb, no more than about 5 kb, or less, in a genomc.

The term “homolog” refers to a gene that is part of a group of genes that are related by descent from a common ancestor (i.e., gene sequences (i.e., nucleic acid sequences) of the group of genes and/or the sequences of their protein products are inherited through a common origin). Homologs may arise through speciation events (giving rise to “orthologs”), through gene duplication events, or through horizontal gene transfer events. Homologs may be identified by phylogenetic methods, through identification of common functional domains in the aligned nucleic acid or protein sequences, or through sequence comparisons.

The term “ortholog” refers to a gene that is part of a group of genes that are predicted to have evolved from a common ancestral gene by speciation.

The terms “bidirectional best hit” and “BBH” are used herein interchangeably to refer to the relationship between a pair of genes in two genomes (i.e., a first gene in a first genome and a second gene is a second genome) wherein the first gene or its protein product has been identified as having the most similar sequence in the first genome as compared to the second gene or its protein product in the second genome, and wherein the second gene or its protein product has been identified as having the most similar sequence in the second genome as compared to the first gene or its protein product in the first genome. The first gene is the bidirectional best hit or BBH of the second gene, and the second gene is the bidirectional best hit of BBH of the first gene. BBH is a commonly used method to infer orthology.

As used herein, “sequence similarity” between two genes means similarity of either the nucleic acid (e.g., mRNA) sequences encoded by the genes or the amino acid sequences of the gene products.

“Percent (%) sequence identity” or “percent (%) sequence homology” with respect to the nucleic acid sequences (or protein sequences) described herein is defined as the percentage of nucleotide residues (or amino acid residues) in a candidate sequence that are identical or homologous with the nucleotide residues (or amino acid residues) in the oligonucleotide (or polypeptide) with which the candidate sequence is being compared, after aligning the sequences and considering any conservative substitutions as part of the sequence identity. Homology between different amino acid residues in a polypeptide is determined based on a substitution matrix, such as the BLOSUM (BLOcks SUbstitution Matrix) matrix. Methods for aligning sequences and determining percent sequence identity or percent sequence homology are well known to those of skill in the art. Examples of publicly available computer software that may be used include, but are not limited to, BLAST (Basic Local Alignment Search Tool; software for comparing the amino-acid sequences of proteins or the nucleotide sequences of DNA and/or RNA molecules), BLAST-2, ALIGN or Megalign (DNASTAR) software. Any of a variety of suitable parameter for measuring sequence alignment and determining percent sequence identity or homology may be determined by those of skill in the art, including use of algorithms required to achieve maximal alignment over the full length of the sequences being compared.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The section headings used herein are for organizational purposes only and are not to be construed as limiting the subject matter described. The description is presented to enable one of ordinary skill in the art to make and use the invention, and is provided in the context of a patent application and its requirements.

### Deep Learning Methods for Biosynthetic Gene Cluster (BGC) Discovery

Genome-encoded molecules (GEMs) are a diverse group of molecules, e.g., natural products or secondary metabolites, that are encoded in the genome and produced by groups of genes known as biosynthetic gene clusters (BGCs) in a variety of eukaryotic and prokaryotic organisms. Due to the unique challenges associated with discovering BGCs that encode for potential drug compounds, most traditional carly drug development approaches continue to rely on in vivo or in silico screening methods for lead compound identification. As a result, GEMs represent an as-yet underutilized resource in drug discovery.

Although some computational methods for BGC discovery based on genomic sequences already exist, the performance of current computational models is far from adequate. Difficulty arises from the high dimensionality of genomic data and the relatively small number of high-quality annotated BGCs available for use as training cases.

To address this difficulty, we have acquired a collection of hundreds of thousands of fungal organisms. Via fermentation and genomic sequencing of these organisms, we have created a genomics database comprising a catalogue of high-quality, annotated fungal genomes. These genomes contain a vast number of BGCs, and as such, represent a rich resource for enabling the creation of novel computational methods for identifying and categorizing BGCs and their associated GEMs. Here, we describe methods for the creation and validation of a novel computational pipeline for the effective discovery of BGCs utilizing recent advances in artificial intelligence and machine learning.

Generation of high-quality datasets and synthetic training genomes for supervised learning: Annotated genomes (e.g., fungal, bacterial, or plant genomes) are acquired from a genomics database. Examples of suitable genomics databases include, but are not limited to, Brassica.info, Ensembl Plants, EnsemblFungi, the National Center for Biotechnology Information (NCBI) whole genome database, the Plant Genome Database Japan's DNA Marker and Linkage database, Phytozome, the Plant GDB Genome Browser, FungiDB, the MycoCosm 1000 Fungal Genomes Project database, the FDBC fungal genome database, the Seoul National University Genome Browser (SNUGB) database, AspGD, etc.

Putative BGC regions are recovered and are manually curated using comparative genomics techniques (sec, e.g., International Patent Application No. PCT/US2022/049016, the contents of which are incorporated herein in their entirety) to identify BGCs with high-confidence boundaries. For each BGC, nucleotide sequences of the constituent genes are translated into corresponding peptide sequences, whose functional or conserved domains are annotated using, for example, sequence alignments against the Pfam database, or via InterProScan sequence alignments against the InterPro database, or using a similar protein domain annotation tool. Each gene in the BGC is thus represented as a domain architecture. The resulting sequence of domain architectures is retained as a positive BGC example for use in generating training data for supervised learning.

Negative BGC examples are created according to the following procedure. Annotated fungal genomes are acquired from a genomics database. Putative BGC regions are removed, creating genome-like sequences devoid of biosynthetic gene cluster content. The remaining genes are translated into peptide sequences, and further processed into sequences of, e.g., Pfam protein domains or InterPro protein domains, as described above. The resulting sequence of domain architectures is referred to as a negative genome. A positive BGC example and a negative genome are selected at random. Each domain architecture in the positive BGC example is replaced with a random domain architecture containing the same number of Pfam domains from the negative genome to create a negative BGC example for use in generating training data for supervised learning.

Two sets of synthetic training genomes are created. For the first set, one or more positive BGC examples selected from a subset of the positive BGC examples is randomly inserted into each negative genome from a subset of the negative genomes to create a training genome. For the second set, all positive and negative BGC examples are combined to create a single training genome. Additional training genomes in this set are created by permuting the ordering of positive and negative BGC examples in the training genome.

Representation of training genomes as feature vectors: A representation learning system can be trained on a corpus of, for example, fungal genomes. To create this corpus, annotated fungal genomes are acquired from a genomics database. For each gene in a genome, we retrieve the protein sequence and annotate it according to protein domains, e.g., Pfam domains, as described above. In doing so, we reduce the genome to an ordered list of protein sequences and their constituent protein domains, e.g., Pfam domains. This corpus may then be used to develop a fungal-specific embedding for genome representation via word2vec, GloVe, fastText, or other self-supervised learning algorithms. This embedding may be further refined by using the resulting representation to train, for example, an autoencoder or other unsupervised learning algorithm. The end result is a representation learning system capable of accepting as input an annotated genome representation and producing as output an embedded representation of the genome as an ordered list of vectors, each of which represents a specific protein domain, e.g., a Pfam domain, or annotation.

In some instances, the representation learning system may be trained on a corpus of plant genomes, fungal genomes, bacterial genomes, or any combination thereof.

In some instances, the protein sequence for each gene in a training genome may be annotated using, e.g., CDD, Gene3D, PANTHER, Pfam, ProSitePatterns, ProSiteProfiles, SUPERFAMILY, SMART, TIGRFAM, SFLD, Hamap, Coils, PRINTS, PIRSR, AntiFam, MobiDBLite, PIRSF, or other representations of protein domains.

In some instances, each training genome may be represented as a sequence of annotations of genes based on, e.g., the KEGG or EggNOG database.

Generation of class labels for supervised learning: For each synthetic training genome, individual annotated protein domains are assigned a class label based on whether it belongs to a positive BGC example within its synthetic training genome. For example, annotated protein domains belonging to a positive BGC example may be assigned a positive class label of 1. Annotated protein domains not belonging to a positive BGC example may be assigned a negative class label of 0. For each training genome, these labels can be appended in order of their respective annotated protein domains to create a target vector (i.e., a vector that defines a list of dependent variables in the training dataset) for supervised learning. Because training genomes are composed of positive BGC examples separated by non-biosynthetic or negative BGC example regions, each target vector contains a mixture of positive and negative class labels.

Training supervised classification models: Supervised machine learning methods, such as deep learning methods, may be applied to create computational models that relate the training genome representations with their associated class labels.

Any of a variety of supervised learning methods known to those of skill in the art may be used. Examples include, but are not limited to deep learning methods based on modern state-of-the-art artificial neural networks, such as convolutional neural networks (CNNs), long short-term memory networks (LSTMs), and transformer models.

Convolutional neural network: Convolutional neural networks are a specialized deep neural network architecture consisting of alternating convolution and max-pooling layers that serve to learn feature representations of the input matrix. Each convolutional layer consists of one or more filters that subdivide the input data matrix row-wise to generate genome-region-specific feature maps. These feature maps are summarized by the max-pooling layer to create a condensed representation of the original input matrix. This process can be repeated if the output of the max-pooling layer becomes the input to another pair of convolution and max-pooling layers. The final max-pooling layer is flattened and serves as the input to a fully connected neural network with an activation function that generates the final classification.

Long short-term memory network: Long short-term memory networks are a specialized recurrent neural network (RNN) architecture consisting of a collection of memory cells connected in sequence. A basic RNN cell accepts a hidden state from the previous cell, combines it with input data in the form of a single row of the input matrix, modifies it via an activation function, and outputs a new hidden state, which is both used to calculate the classification of the row, and passed to the next RNN cell, which proceeds to process the next row of input data. An LSTM cell performs the same basic function, but maintains an additional representation known as the cell state and contains additional connections and activation functions that enable decision-making to keep or forget information. A forget gate takes as input the previous hidden state and the new input data, and applies an activation function to determine which information to forget. This is used to modify the previous cell state, overwriting data to be forgotten with a 0. The input gate takes as input the previous hidden state and the new input data, and applies an activation function to determine which information to update. A separate activation function is applied to determine the actual values of the updated information. These two activations are combined and used to update the cell state from the forget gate. Finally, an output gate combines activations from the hidden state and the updated cell state to determine the new hidden state. As in an RNN cell, the hidden state is used to calculate the classification, and both the new hidden state and updated cell state are passed to the next cell. LSTMs can be unidirectional, consisting of one sequence of LSTM cells connected in sequence, or bidirectional, containing two chains of LSTM cells connected in opposing directions.

Transformer models: Transformers are a state-of-the-art neural network architecture that enables parallelization via the introduction of a self-attention mechanism, removing the sequential dependency of RNNs. A transformer consists of a stack of encoders and a stack of decoders. An encoder consists of a self-attention layer and a feed-forward neural network. A decoder contains both of these components as well, but also contains an encoder-decoder attention layer, to accept and focus input from the final encoder layer. During training, the entire input matrix is used to determine the self-attention values, but the feed-forward networks are evaluated individually for each row. The output of the final decoder layer is used for classification.

In some instances, unsupervised machine learning approaches may be used to implement the disclosed methods for identifying the genes belonging to a biosynthetic gene cluster in an input genome. Unsupervised machine learning is used to identify patterns in training datasets containing data points that are neither classified nor labeled. Examples of unsupervised machine learning models that may be used include, but are not limited to, generative models such as variational autoencoders, flow-based models, diffusion models, and generative adversarial models, or non-generative methods such as clustering or traditional autoencoders.

Regardless of the model architecture, each machine learning method produces a computational model that is capable of accepting an encoded representation, e.g., an encoded Pfam representation, of a new genome and returning a vector containing an annotation describing whether or not the encoded protein domain representations belong to a BGC. Depending on model performance, these solutions may be applied independently, applied in sequence, or further integrated via an ensemble learning technique such as bagging, boosting, or related methods.

Training machine learning models: The weighting factors, bias values, and threshold values, or other computational parameters of a machine learning model, e.g., a neural network, can be “taught” or “learned” in a training phase using one or more sets of training data and any of a variety of training methods known to those of skill in the art. For example, the parameters for a neural network may be trained using the input data from a training data set and a gradient descent or backward propagation method so that the output predictions (e.g., predictions of the presence of a biosynthetic gene cluster (BGC) in a genome) of the trained neural network are consistent with the examples included in the training data set. The adjustable parameters of, e.g., a neural network, model may be obtained from a back propagation neural network training process that may or may not be performed using the same hardware as that used for processing genomic data during a deployment phase.

Training data sets: In some instances, as described above, the training data used to train a machine learning model of the present disclosure may comprise representations of one or more synthetic training genomes (e.g., 1, 2, 3, 4, 5, 6, 7, 8, 9, 10, 20, 30, 40, 50, 60, 70, 80, 90, 100, 200, 300, 400, 500, 600, 700, 800, 900, 1000, 10,000, 100,000 or more than 100,000 synthetic training genomes, or any number of synthetic training genomes within this range). In some instances, e.g., if using a supervised learning approach, the training data may comprise labeled training data, e.g., labeled representations of one or more synthetic training genomes. In some instances, e.g., if using an unsupervised learning approach, the training data may comprise unlabeled training data, e.g., unlabeled representations of one or more synthetic training genomes. In some instances, one or more training data sets may be used to train the machine learning algorithm in a training phase that is distinct from that of the deployment or use phase. In some instances, the training data may be continuously updated, and used to update the trained machine learning algorithm in real time. In some cases, the training data may be stored in a training database that resides on a local computer or server. In some cases, the training data may be stored in a training database that resides online or in the cloud.

Machine learning software: Any of a variety of commercial or open-source software packages, software languages, or software platforms known to those of skill in the art may be used to implement the machine learning algorithms of the disclosed methods and systems. Examples include, but are not limited to, Shogun (www.shogun-toolbox.org), Mlpack (www.mlpack.org), R (r-project.org), Weka (www.cs.waikato.ac.nz/ml/weka/),Python (www.python.org), Matlab (MathWorks, Natick, MA, www.mathworks.com), scikit-learn (www.scikit-learn.org), tensorflow (www.tensorflow.org), pytorch (www.pytorch.org), and/or keras (www.keras.io).

Distributed computing systems and cloud-based training databases: In some instances, the machine learning-based methods for identifying biosynthetic gene clusters (BGCs) disclosed herein may be used for processing genomic data (e.g., sequence data) on one or more computers or computer systems that reside at a single physical or geographical location. In some instances, they may be deployed as part of a distributed system of computers that comprises two or more computer systems residing at two or more physical or geographical locations. Different computer systems, or components or modules thereof, may be physically located in different workspaces and/or different worksites (i.e., in different physical or geographical locations), and may be linked via a local area network (LAN), an intranet, an extranet, or the Internet so that training data and/or data from processing input genomes may be shared and exchanged between the sites.

In some embodiments, training data (e.g., comprising synthetic training genome data) may reside in a cloud-based database that is accessible from local and/or remote computer systems on which the disclosed machine learning-based methods are running. As used herein, the term “cloud-based” refers to shared or sharable storage of electronic data. The cloud-based database and associated software may be used for archiving electronic data, sharing electronic data, and analyzing electronic data.

In some embodiments, training data (e.g., comprising synthetic training genome data) generated locally may be uploaded to a cloud-based database, from which it may be accessed and used to train other machine learning-based systems at the same site or at a different site. In some instances, machine learning-based prediction results (e.g., detection of patterns of predicted protein domains encoded by genes belonging to a biosynthetic gene cluster (BGC), and identification of the genes in the cluster) generated locally may be uploaded to a cloud-based database and used to update the training data set in real time for continuous improvement of prediction performance.

Internal and external validation: Model performance may be evaluated internally using, for example, a k-fold cross validation method. In this validation framework, the training dataset is randomly split into k groups, where k can range from 2 to n, and where n is the number of training samples. In some instances, for example, n may be 2, 3, 4, 5, 6, 7, 8, 9, 10, or more than 10. In some instances, the machine learning model is trained on k-1 groups, and then the performance of this trained model is evaluated on the remaining group. This is done k times, such that each group serves as the validation group once, and as part of the external training group k-1 times. Performance metrics across the k folds can then be aggregated.

External validation is performed by testing model performance on a gold-standard set of, e.g., fungal genomes that have been manually reviewed and annotated for BGCs. In both internal and external validation, model performance is evaluated using typical information retrieval metrics, such as precision and recall (where precision quantifies the number of positive class predictions that actually belong to the positive class, and recall quantifies the number of positive class predictions made for all positive examples in the validation data). In some instances, precision and/or recall may be at least 0.5, at least 0.6, at least 0.7, at least 0.75, at least 0.8, at least 0.85, at least 0.9, at least 0.95, at least 0.98, or at least 0.99.

Prediction of novel fungal BGCs: Just as the disclosed machine learning models can be used to annotate well-studied genomes to evaluate their ability to recover known BGCs, they can also be applied to new genomes to identify novel BGCs. These novel BGCs can then be classified, e.g., according to function and the class of compound produced by the BGC, using additional computational methods.

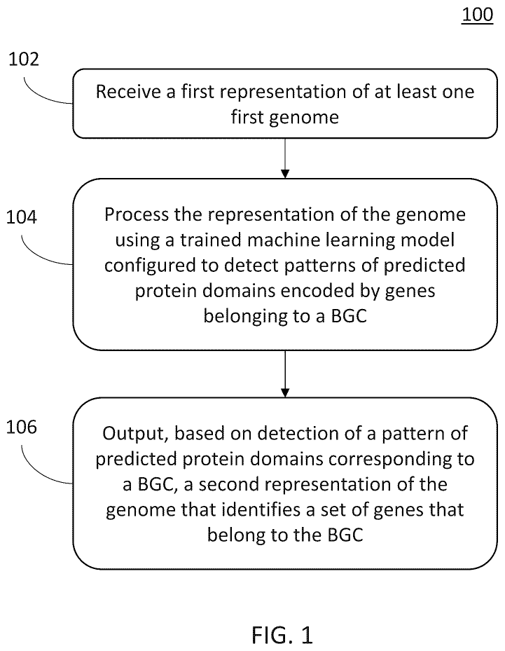

FIG. 1 provides a non-limiting example of a flowchart for a process 100 for predicting genes that belong to a biosynthetic gene cluster (BGC) in a first representation of a genome. Process 100 can be performed, for example, as a computer-implemented method using software running on one or more processors of one or more electronic devices, computers, or computing platforms. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

At step 102 in FIG. 1, a first representation of at least one first genome is received. For example, the first representation of the at least one first genome may be input by a user of a system configured to perform the computer-implemented methods described herein.

In some instances, the at least one first genome may comprise a eukaryotic genome or a prokaryotic genome. In some instances, the at least one first genome may be a eukaryotic genome, and the eukaryotic genome may comprise a plant genome or a fungal genome. In some instances, the at least one first genome may be a prokaryotic genome, and the prokaryotic genome may be a bacterial genome.

In some instances, the at least one first genome may comprise 1, 2, 3, 4, 5, 6, 7, 8, 9, 10, 20, 30, 40, 50, 60, 70, 80, 90, 100, 200, 400, 600, 800, 1,000, or more than 1,000 first (or input) genomes, or any number of first (or input) genomes within this range.

In some instances, the first representation of the at least one first genome may comprise a nucleotide sequence for the at least one first genome. In some instances, the first representation of the at least one first genome may comprise a vector representation of the at least one first genome, or an embedding thereof.

In some instances, the first representation of the at least one first genome may comprise a sequence of CDD, Gene3D, PANTHER, Pfam, ProSitePatterns, ProSiteProfiles, SUPERFAMILY, SMART, TIGRFAM, SFLD, Hamap, Coils, PRINTS, PIRSR, AntiFam, MobiDBLite, or PIRSF representations, or any combination thereof, of protein domains encoded by genes within the at least one first genome. In some instances, the sequence of protein domain representations is generated by a process comprising retrieving a protein sequence for each gene in the at least one first genome and identifying protein domains in the protein sequence by sequence alignment against a database of CDD, Gene3D, PANTHER, Pfam, ProSitePatterns, ProSiteProfiles, SUPERFAMILY, SMART, TIGRFAM, SFLD, Hamap, Coils, PRINTS, PIRSR, AntiFam, MobiDBLite, or PIRSF representations, respectively. In some instances, the first representation of the at least one first genome may further comprise associated gene ontology (GO) terms, an identification of any resistance genes that are present in the at least one first genome, an identification of additional regulatory elements such as promoters, enhancers, or silencers, that are present in the at least one first genome, or an identification of additional epigenetic elements, such as histone folding, DNA methylation or acetylation, that are present in the at least one first genome.

In some instances, the method may further comprise encoding each protein domain representation in the sequence of CDD, Gene3D, PANTHER, Pfam, ProSitePatterns, ProSiteProfiles, SUPERFAMILY, SMART, TIGRFAM, SFLD, Hamap, Coils, PRINTS, PIRSR, AntiFam, MobiDBLite, or PIRSF representations of protein domains as a vector representation of the at least one first genome using a representation learning system. In some instances, the representation learning system comprises a graphical learning model, a deep autoencoder, Pfam2vec, word2vec, GloVe, or fastText.

In some instances, the first representation of the at least one first genome may comprise a sequence of annotations of genes within the at least one first genome based on a gene function and pathway mapping database. In some instances, for example, the pathway mapping database may be KEGG.

In some instances, the first representation of the at least one first genome may comprise a sequence of annotations of genes within the at least one first genome based on a database comprising data for clusters of orthologous groups (COGs). In some instances, for example, the database comprising data for clusters of orthologous groups (COGs) is EggNOG.

At step 104 in FIG. 1, the representation of the at least one first genome is processed using a trained machine learning model configured to detect patterns of predicted protein domains encoded by genes belonging to a biosynthetic gene cluster (BGC).

In some instances, the trained machine learning model may comprise a deep learning model. For example, the deep learning model may comprise a supervised learning model (e.g., a supervised deep learning model) or an unsupervised learning model (e.g., an unsupervised deep learning model). In some instances, the deep learning model comprises a convolutional neural network, a long short-term memory network, or a transformer model. In some instances, the deep learning model may comprise a combination of components from a neural network, a convolutional neural network, a long short-term memory network, or a transformer neural network.

In some instances, the machine learning model may be trained using a training data set comprising data for (e.g., representations of) a plurality of training genomes. In some instances, the plurality of training genomes may comprise a plurality of synthetic training genomes. In some instances, one or more synthetic training genomes of the plurality of synthetic training genomes may each comprise a set of gene sequences from an actual BGC randomly inserted into a BGC negative genome. In some instances, one or more synthetic training genomes of the plurality of synthetic training genomes may each comprise a set of gene sequences from a combination of actual BGCs and artificially-generated non-BGCs.

In some instances, the training data set may comprise data for (e.g., representations of) at least 5, 10, 20, 30, 40, 50, 60, 70, 80, 90, 100, 1000, 10,000, 100,000, or more than 100,000 training genomes (e.g., data for (e.g., representations of) at least 5, 10, 20, 30, 40, 50, 60, 70, 80, 90, 100, 1000, 10,000, 100,000, or more than 100,000 synthetic training genomes), or any number of training genomes (or synthetic training genomes) within this range.

At step 106 in FIG. 1, a second representation of the at least one first genome that identifies a set of genes that belong to the BGC is output based on detection of a pattern of predicted protein domains corresponding to a BGC in the first representation of the at least one first genome. In some instances, the second representation of the at least one first genome comprises a vector representation, a graph representation, or a tensor representation of the at least one first genome.

In some instances, the computer-implemented method may further comprise evaluating a gene identified as belonging to the BGC to determine if it is a resistance gene. In some instances, the resistance gene may be an embedded target gene (ETaG) or a non-embedded target gene (NETaG).

In some instances, the methods described herein may further comprise using the output of the computer-implemented method (e.g., an identification of a resistance gene associated with a BGC) to perform an in vitro assay to test a secondary metabolite produced by the BGC in the at least first genome to which the resistance gene has been identified as belonging for activity against a resistance gene homolog, or protein encoded thereby, identified in a second genome that differs from the at least one first genome.

In some instances, the methods described herein may further comprise using the output of the computer-implemented method (e.g., an identification of a resistance gene associated with a BGC) to perform an in vivo assay to test a secondary metabolite produced by the BGC in the at least first genome to which the resistance gene has been identified as belonging for activity against a resistance gene homolog, or protein encoded thereby, identified in a second genome that differs from the at least one first genome.

In some instances, the second genome may comprise a mammalian genome, a human genome, an avian genome, a reptilian genome, an amphibian genome, a plant genome, a fungal genome, a bacterial genome, or a viral genome.

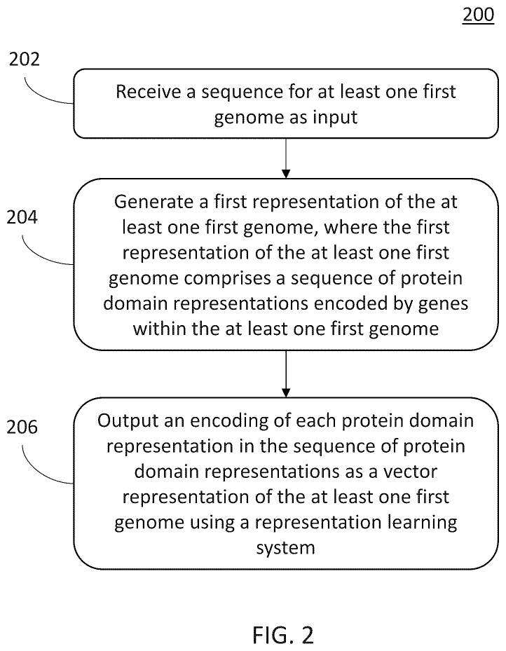

FIG. 2 provides a non-limiting example of a process flowchart for generating an embedded representation of a genome as an ordered list of vectors, each of which represents a specific protein domain, e.g., a Pfam domain, or annotation.

At step 202 in FIG. 2, a sequence for at least first genome is received as input. In some instances, the at least one first genome may be input by a user of a system configured to perform the computer-implemented methods described herein.

In some instances, the at least one first genome may comprise a eukaryotic genome or a prokaryotic genome as described elsewhere herein. In some instances, the at least one first genome may be a eukaryotic genome, and the eukaryotic genome may comprise a plant genome or a fungal genome. In some instances, the at least one first genome may be a prokaryotic genome, and the prokaryotic genome may be a bacterial genome.

In some instances, the at least one first genome may comprise 1, 2, 3, 4, 5, 6, 7, 8, 9, 10, 20, 30, 40, 50, 60, 70, 80, 90, 100, 200, 400, 600, 800, 1,000, or more than 1,000 first (or input) genomes, or any number of first (or input) genomes within this range.

At step 204 in FIG. 2, a first representation of the at least one first genome is generated, where the first representation of the at least one first genome comprises a sequence of protein domain representations, e.g., Pfam domains or other protein domain representations, encoded by genes within the at least one first genome.

In some instances, the first representation of the at least one first genome may comprise a sequence of CDD, Gene3D, PANTHER, Pfam, ProSitePatterns, ProSiteProfiles, SUPERFAMILY, SMART, TIGRFAM, SFLD, Hamap, Coils, PRINTS, PIRSR, AntiFam, MobiDBLite, or PIRSF representations, or any combination thereof, of protein domains encoded by genes within the at least one first genome, as described elsewhere herein. In some instances, the sequence of protein domain representations is generated by a process comprising retrieving a protein sequence for each gene in the at least one first genome and identifying protein domains in the protein sequence by sequence alignment against a database of CDD, Gene3D, PANTHER, Pfam, ProSitePatterns, ProSiteProfiles, SUPERFAMILY, SMART, TIGRFAM, SFLD, Hamap, Coils, PRINTS, PIRSR, AntiFam, MobiDBLite, or PIRSF representations, respectively. In some instances, the first representation of the at least one first genome may further comprise associated gene ontology (GO) terms, an identification of any resistance genes that are present in the at least one first genome, an identification of additional regulatory elements such as promoters, enhancers, or silencers, that are present in the at least one first genome, or an identification of additional epigenetic elements, such as histone folding, DNA methylation or acetylation, that are present in the at least one first genome.

At step 206 in FIG. 2, an encoding of each protein domain representation in the sequence of protein domain representations as a vector representation of the at least one first genome using a representation learning system is output.

In some instances, as described elsewhere herein, the representation learning system may comprise a graphical learning model, a deep autoencoder, Pfam2vec, word2vec, GloVe, or fastText.

In some instances, as described elsewhere herein, the representation learning system may be trained on a corpus of annotated genomes (e.g., annotated fungal genomes), cach comprising a sequence of CDD, Gene3D, PANTHER, Pfam, ProSitePatterns, ProSiteProfiles, SUPERFAMILY, SMART, TIGRFAM, SFLD, Hamap, Coils, PRINTS, PIRSR, AntiFam, MobiDBLite, or PIRSF representations, or any combination thereof, of protein domains encoded by genes within a genome of the corpus.

### Using Random Forests to Predict Compound Class and Function in Novel Fungal BGCs

Generation of class labels for supervised learning: Biosynthetic product class and compound activities for bacterial and fungal BGCs for use as training data may be obtained in JSON format from, e.g., the MIBiG database (Version 2.0; https://mibig.secondarymetabolites.org/). Product classes are taken from the “biosyn_class” field and compound activities are taken from the “chem_act” field. Product classes describe the molecular type of GEM associated with the BGC and contains classifications such as polyketide, saccharide, or non-ribosomal peptide. Compound activity describes the chemical activity of the GEM associated with the BGC and contains annotations such as antibacterial, antifungal, or cytotoxic. A BGC may belong to more than one product class or have more than one type of compound activity. BGCs with no known activity or product class are omitted from the training set. Each BGC is assigned a label vector where each element of the vector corresponds to a unique product class or chemical activity. Elements of the label vector are marked with a 1 if the BGC produces that product class or the product has the corresponding activity, and 0 otherwise.

Representation of BGCs as feature vectors: For each gene in a BGC, we translate the nucleotide sequence to a corresponding peptide sequence and identify the predicted protein domains. For example, Pfam protein domains may be identified using InterProScan. These gene in the BGC can then be further described as the combination of Pfam domains they contain in order from start to end. These combinations are referred to as domain architectures. In addition, we annotate the BGC genes with associated gene ontology (GO) terms and the presence of any resistance genes or additional regulatory or epigenetic elements.

To represent these annotations as input vectors we create unique input matrices consisting of individual representation schemes or in combinations such as but not limited to:
copy number of Pfam domains (or other protein domain representations) and resistance genes
Copy number of GO terms
Copy number of domain architectures
Copy number of GO terms and domain architectures

Training Random Forests: For a given input matrix, each feature vector is mapped to its corresponding label vector. Using each input matrix, separate random forest classification models are trained to perform multi-label classification on product classes, multi-label classification on chemical activities, binary classification for each product class, and binary classification for each chemical activity.

Model performance may be evaluated with a cross-validation framework as described above. Feature selection is performed through recursive feature elimination of features with low or null contribution scores as measured by the GINI criterion. Class imbalance for binary classification is addressed through down-sampling of the majority class in the training sets, creating an ensemble of models, and evaluated on an additional validation set held in reserve.

This process may be performed using, e.g., bacterial BGCs only, fungal BGCs, and fungal+bacterial BGCs to identify the best model for each classification task.

Identification of feature combinations important for classification: Logical rules in the form of “If x>1 AND y 21 =3 AND z<4 then predict Class A” are identified for each classification task until the full training set is accounted for by a compact set of rules. Rules are identified by traversing the structure of the final tree-based model and greedily re-constructing the minimal set of conditions that associate a BGC with its correct label subject to regularization. For each classification task the optimal set of association rules is identified using the Certified Optimal Rule Lists (CORELS) algorithm.

Alternative Methods: Existing methods for BGC identification from genomes include ClusterFinder, antiSMASH, DeepBGC, and TOUCAN. ClusterFinder employs a hidden Markov model (HMM) trained on a collection of bacterial BGCs. DeepBGC utilizes a bi-directional LSTM, also trained on bacterial BGCs. Both solutions, when applied to identify BGCs in fungi, perform poorly. antiSMASH consists of a rule-based expert system that integrates data from several different profile hidden Markov models, and is the current standard approach for BGC discovery. TOUCAN is a combination framework that utilizes three support vector machines, a multilayer perceptron, logistic regression, and random forest algorithms. However, it does not contain functionality for combining predictions from these different methods into a single output.

### Applications

The computer-based methods for predicting the presence of BGCs and identifying their associated genes as described herein have various applications including, for example, performing further evaluation of the genes predicted to be part of a BGC to: (i) identify homologs or orthologs of one or more target sequences (e.g., gene sequences) of interest in one or more target genomes, (ii) identify a resistance gene against a secondary metabolite produced by a BGC in a target genome, (iii) predict a function of a secondary metabolite produced by a BGC, and/or (iv) identify a BGC that encodes biosynthetic enzymes for producing a secondary metabolite having an activity of interest (e.g., a therapeutic activity of interest), etc.

Methods for evaluating genes embedded within or associated with a BGC to identify resistance genes (e.g., “embedded target genes” (ETaGs) or “non-embedded target genes” (NETaGs)) have been described in International Patent Application Nos. PCT/US2022/049016, PCT/US2022/049040, and PCT/US2022/079965, the contents of each of which are incorporated herein in their entirety. In some instances, for example, a method for identifying resistance genes (e.g., embedded target genes (ETaGs) and/or non-embedded target genes (NETaGs)) may comprise: receiving a selection of at least one target sequence of interest; receiving a selection of target genomes from a genomics database, wherein the selection of target genomes comprises a plurality of target genomes from organisms that are known to produce, or are likely to produce, secondary metabolites; performing a search to identify homologs of the at least one target sequence in the plurality of target genomes; generating a phylogenetic tree based on the identified homologs of the at least one target sequence; classifying the genomes of the plurality of target genomes as positive genomes or negative genomes based on the phylogenetic tree, wherein positive genomes are genomes that belong to a clade for which multiple copies of the at least one target sequence homolog are present, wherein negative genomes are genomes that belong to a clade for which a single copy of the at least one target sequence homolog is present; and wherein a target sequence homolog that is present in multiple copies in a positive genome is a putative resistances genes (e.g., a pETaG or pNETaG); determining, based at least in part on the classification of positive and negative genomes, at least one genomic parameter selected from the following: i) one or more scores indicative of co-occurrence of the at least one target sequence homolog (putative resistance gene (e.g., a pETaG or pNETaG)) and one or more genes associated with a biosynthetic gene cluster (BGC); ii) one or more scores indicative of co-evolution of the at least one target sequence homolog (putative resistance gene (e.g., a pETaG or pNETaG)) and one or more genes associated with a BGC; iii) one or more scores indicative of co-regulation of the at least one target sequence homolog (putative resistance gene (e.g., a pETaG or pNETaG)) with one or more genes associated with a BGC; and iv) one or more scores indicative of co-expression of the at least one target sequence homolog (putative resistance gene (e.g., a pETaG or pNETaG)) with one or more genes associated with a BGC; and determining, based on the at least one genomic parameter, a likelihood that the putative resistance gene (e.g., pETaG or pNETaG) is a resistance gene (e.g., an embedded target gene (ETaG) or non-embedded target gene (NETaG)).

In some instances, determining the likelihood that the putative resistance gene is a resistance gene may comprise comparing the at least one determined genomic parameter to at least one predetermined threshold.

In some instances, the selection of at least one target sequence of interest may be provided as input by a user of a system configured to perform the computer-implemented method. In some instances, for example, the at least one target sequence of interest may comprise a sequence of a gene identified as belonging to a BGC by any of the methods described elsewhere herein.

In some instances, the at least one target sequence of interest may comprise an amino acid sequence, a nucleotide sequence, or any combination thereof. In some instances the at least one target sequence of interest may comprise a peptide sequence or portion thereof, a protein sequence or portion thereof, a protein domain sequence or portion thereof, a gene sequence or portion thereof, or any combination thereof. In some instances, the at least one target sequence of interest may comprise a mammalian sequence, a human sequence, a plant sequence, a fungal sequence, a bacterial sequence, an archaea sequence, a viral sequence, or any combination thereof.

In some instances, the at least one target sequence of interest may comprise a primary target sequence and one or more related sequences. In some instances, the one or more related sequences may comprise sequences that are functionally-related to the primary target sequence. In some instances, the one or more related sequences may comprise sequences that are pathway-related to the primary target sequence.

In some instances, the selection of target genomes may be provided as input by a user of a system configured to perform the computer-implemented method. In some instances, the plurality of target genomes may comprise plant genomes, fungal genomes, bacterial genomes, or any combination thereof. In some instances, the genomics database may comprise a public genomics database. In some instances, the genomics database comprises a proprietary genomics database.

In some instances, the search to identify homologs of the at least one target sequence (e.g., homologs of a gene sequence identified as belonging to a BGC) may comprise identification of homologs based on probabilistic sequence alignment models. In some instances, the probabilistic sequence alignment models are profile hidden Markov models (pHMMs). In some instances, homologs are identified based on a comparison of probabilistic sequence alignment model scores to a predefined threshold.

In some instances, the search to identify homologs of the at least one target sequence may comprise identification of homologs based on alignment of sequences using a local sequence alignment search tool, calculation of a sequence homology metrics based on the alignments, and comparison of the calculated sequence homology metrics to a predefined threshold. In some instances, the local sequence alignment search tool comprises BLAST, DIAMOND, HMMER, Exonerate, or ggsearch. In some instances, the predefined threshold may comprise a threshold for percent sequence identity, percent sequence coverage, E-value, or bitscore value.

In some instances, the search to identify homologs of the at least one target sequence may comprise identification of homologs based on use of a gene and/or protein domain annotation tool. In some instances, the gene and/or protein domain annotation tool comprises InterProScan or EggNOG.

In some instances, the generation of phylogenetic trees based on the identified homologs of the at least one target sequence may comprise alignment of homolog sequences using an alignment software tool, trimming of the aligned homolog sequences using a sequence trimming software tool, and construction of a phylogenetic tree using phylogenetic tree building software tool. In some instances, the alignment software tool comprises MAFFT, MUSCLE, or ClustalW. In some instances, the sequence trimming software tool comprises trimAI, GBlocks, or ClipKIT.

In some instances, the phylogenetic tree building software tool comprises FastTree, IQ-TREE, RAXML, MEGA, MrBayes, BEAST, or PAUP. In some instances, the construction of the phylogenetic tree may be based on a maximum likelihood algorithm, parsimony algorithm, neighbor joining algorithm, distance matrix algorithm, or Bayesian inference algorithm.

In some instances, the one or more scores indicative of co-occurrence may be determined based on identifying positive correlations between the presence of multiple copies of a putative resistance gene and the presence of the one or more genes of a BGC in positive genomes. In some instances, identifying the positive correlations between the presence of multiple copies of the putative resistance gene and the presence of the one or more genes of a BGC in positive genomes may comprise the use of a clustering algorithm to cluster aligned protein sequences, aligned nucleotide sequences, aligned protein domain sequences, or aligned pHMMs for a group of BGCs to identify BGC communities within the plurality of target genomes. In some instances, identifying the positive correlations between the presence of multiple copies of the putative resistance gene and the presence of the one or more genes of a BGC in positive genomes may comprise the use of a phylogenetic analysis of protein sequences or protein domains for a group of BGCs to identify BGC communities within the plurality of target genomes. In some instances, identifying the positive correlations between the presence of multiple copies of the putative resistance gene and the presence of the one or more genes of a BGC in positive genomes may comprise choosing genomes with a specific taxonomy to identify BGC communities within the plurality of target genomes.

In some instances, the one or more scores indicative of co-evolution of a putative resistance gene and the one or more genes associated with a BGC may be determined based on a co-evolution correlation score, a co-evolution rank score, a co-evolution slope score, or any combination thereof. In some instances, the co-evolution correlation score may be based on a correlation between pairwise percent sequence identities of a cluster of orthologous groups (COG) for the putative resistance gene and pairwise percent sequence identities of a cluster of orthologous groups (COG) for one of the one or more genes associated with a BGC. In some instances, the co-evolution rank score may be based on a ranking of a correlation coefficient of a COG that contains one of the one or more genes associated with a BGC in ascending order in relation to a COG that contains the putative resistance gene. In some instances, in the case of ties for a distance score, the rank for all COGs in the tie may be set equal to a lowest rank in the group. In some instances, the co-evolution slope score may be based on an orthogonal regression of pairwise percent sequence identities of a COG for the putative resistance gene and pairwise percent sequence identities of a COG for one of the one or more genes associated with a BGC. In some instances, only COGs arising from unique positive genomes that have more than three genes remaining after removing corresponding genes from negative genomes are used to evaluate a co-evolution correlation score, a co-evolution rank score, or a co-evolution slope score.

In some instances, the one or more scores indicative of co-regulation may be based on DNA motif detection from intergenic sequences of the one or more genes associated with a BGC and the putative resistance gene.

In some instances, the one or more scores indicative of co-expression may be based on a differential expression analysis and/or a clustering analysis of global transcriptomics data.

In some instances, the one or more genes associated with a biosynthetic gene cluster (BGC) may comprise an anchor gene, a core synthase gene, a biosynthetic gene, a gene not involved in the biosynthesis of a secondary metabolite produced by the BGC, or any combination thereof.

In some instances, the putative resistance gene may be a putative embedded target gene (pETaG) or a putative non-embedded target gene (pNETaG).

In some instances, the resistance gene may be an embedded target gene (ETaG) or a non-embedded target gene (NETaG).

In some instances, a method for predicting a function of a secondary metabolite may comprise: receiving a selection of at least one target sequence of interest, wherein the at least one target sequence of interest corresponds to a gene sequence associated with a biosynthetic gene cluster (BGC) known to produce the secondary metabolite; receiving a selection of target genomes from a genomics database, wherein the selection of target genomes comprises a plurality of target genomes from organisms that are known to produce secondary metabolites; performing a search to identify homologs of the at least one target sequence in the plurality of target genomes; generating a phylogenetic tree based on the identified homologs of the at least one target sequence; classifying the genomes of the plurality of target genomes as positive genomes or negative genomes based on the phylogenetic tree, wherein positive genomes are genomes that belong to a clade for which multiple copies of the at least one target sequence homolog are present, wherein negative genomes are genomes that belong to a clade for which a single copy of the at least one target sequence homolog is present; and wherein a target sequence homolog that is present in multiple copies in a positive genome is a putative resistance gene; determining, based at least in part on the classification of positive and negative genomes, at least one genomic parameter selected from the following: i) one or more scores indicative of co-occurrence of the at least one target sequence homolog (putative resistance gene) and one or more genes associated with the BGC; ii) one or more scores indicative of co-evolution of the at least one target sequence homolog (putative resistance gene) and one or more genes associated with the BGC; iii) one or more scores indicative of co-regulation of the at least one target sequence homolog (putative resistance gene) with one or more genes associated with the BGC; and iv) one or more scores indicative of co-expression of the at least one target sequence homolog (putative resistance gene) with one or more genes associated with the BGC; and determining, based on the at least one genomic parameter, a likelihood that the putative resistance gene is a resistance gene that encodes a protein target that is acted upon by the secondary metabolite.

In some instances, a method for identifying a biosynthetic gene cluster (BGC) that encodes biosynthetic enzymes for producing a secondary metabolite having an activity of interest may comprise: receiving a selection of at least one target sequence of interest, wherein the at least one target sequence of interest comprises a sequence that encodes a therapeutic target of interest; receiving a selection of target genomes from a genomics database, wherein the selection of target genomes comprises a plurality of target genomes from organisms that are known to produce secondary metabolites; performing a search to identify homologs of the at least one target sequence in the plurality of target genomes; generating a phylogenetic tree based on the identified homologs of the at least one target sequence; classifying the genomes of the plurality of target genomes as positive genomes or negative genomes based on the phylogenetic tree, wherein positive genomes are genomes that belong to a clade for which multiple copies of the at least one target sequence homolog are present, wherein negative genomes are genomes that belong to a clade for which a single copy of the at least one target sequence homolog is present; and wherein a target sequence homolog that is present in multiple copies in a positive genome is a putative resistance gene; determining, based at least in part on the classification of positive and negative genomes, at least one genomic parameter selected from the following: i) one or more scores indicative of co-occurrence of the at least one target sequence homolog (putative resistance gene) and one or more genes associated with a biosynthetic gene cluster (BGC); ii) one or more scores indicative of co-evolution of the at least one target sequence homolog (putative resistance) and one or more genes associated with a BGC; iii) one or more scores indicative of co-regulation of the at least one target sequence homolog (putative resistance gene) with one or more genes associated with a BGC; and iv) one or more scores indicative of co-expression of the at least one target sequence homolog (putative resistance gene) with one or more genes associated with a BGC; and determining, based on the at least one genomic parameter, a likelihood that the putative resistance gene is an actual resistance gene associated with a BGC that produces a secondary metabolite that acts upon a protein product encoded by the resistance gene.

In some instances, the methods of the present disclosure may further comprise performing an in vitro assay, for example, an assay to detect or measure an activity (e.g., a receptor binding activity, an enzyme activation activity, an enzyme inhibition activity, etc.) of a secondary metabolite (or analog thereof) on a mammalian (e.g., human) protein encoded by a mammalian (e.g., human) gene that is homologous to an ETaG or NETaG identified in an organism comprising a biosynthetic gene cluster (BGC) that produces the secondary metabolite. In some instances, the methods may further comprise performing an in vitro assay to detect or measure an activity (e.g., a receptor binding activity, an enzyme activation activity, an enzyme inhibition activity, etc.) of a secondary metabolite (or analog thereof) on a protein (e.g., a reptilian, avian, amphibian, plant, fungal, bacterial, or viral protein) encoded by a reptilian, avian, amphibian, plant, fungal, bacterial, or viral gene that is homologous to an ETaG or NETag identified in an organism comprising a biosynthetic gene cluster (BGC) that produces the secondary metabolite.

In some instances, the methods of the present disclosure may further comprise performing an in vivo assay, for example, an assay to detect or measure an activity (e.g., a receptor binding activity, an enzyme activation activity, an enzyme inhibition activity, an intracellular signaling pathway activity, a disease response, etc.) of a secondary metabolite (or analog thereof) on a mammalian (e.g., human) protein encoded by a mammalian (e.g., human) gene that is homologous to an ETaG or NETaG identified in an organism comprising a biosynthetic gene cluster (BGC) that produces the secondary metabolite. In some instances, the methods may further comprise performing an in vivo assay to detect or measure an activity (e.g., a receptor binding activity, an enzyme activation activity, an enzyme inhibition activity, an intracellular signaling pathway activity, a disease response, etc.) of a secondary metabolite (or analog thereof) on a protein (e.g., a reptilian, avian, amphibian, plant, fungal, bacterial, or viral protein) encoded by a reptilian, avian, amphibian, plant, fungal, bacterial, or viral gene that is homologous to an ETaG or NETaG identified in an organism comprising a biosynthetic gene cluster (BGC) that produces the secondary metabolite.

In some instances, the methods of the present disclosure may be used, for example, for identifying and/or characterizing a mammalian (e.g., human) target of a secondary metabolite (or analog thereof) produced by a BGC. In some instances, the methods of the present disclosure may be used for identifying and/or characterizing a reptilian, avian, amphibian, plant, fungal, bacterial, viral target of a secondary metabolite (or analog thereof) produced by a BGC, or a target from any other organism.

In some instances, the methods of the present disclosure may be used, for example, for drug discovery activities, e.g., to identify small molecule modulators of a mammalian (e.g., human) target gene. In some instances, the methods of the present disclosure may be used to identify small molecule modulators of a reptilian target gene, an avian target gene, an amphibian target gene, a plant target gene, a fungal target gene, a bacterial target gene, a viral target gene, or a target gene from any other organism.

In some instances, the secondary metabolite is a product of enzymes encoded by the BGC or a salt thereof, including an unnatural salt. In some instances, the secondary metabolite or analog thereof is an analog of a product of enzymes encoded by the BGC, e.g., a small molecule compound having the same core structure as the secondary metabolite, or a salt thereof.

In some instances, the present disclosure provides methods for modulating a human target (or a target from another organism), comprising: providing a secondary metabolite produced by enzymes encoded by a BGC, or an analog thereof, wherein the human target (or a nucleic acid sequence encoding the human target) is homologous to an ETaG or NETaG that is associated with the BGC as determined using any one of the methods described herein.

In some instances, the present disclosure provides methods for treating a condition, disorder, or disease associated with a human target (or a target from another organism), comprising administering to a subject susceptible to, or suffering therefrom, a secondary metabolite produced by enzymes encoded by a BGC, or an analog thereof, wherein the human target (or a nucleic acid sequence encoding the human target) is homologous to an ETaG or NETaG that is associated with the BGC as determined using any one of the methods described herein.

In some instances, the secondary metabolite is produced by a fungus. In some instances, the secondary metabolite is acyclic. In some instances, the secondary metabolite is a polyketide. In some instances, the secondary metabolite is a terpene compound. In some instances, the secondary metabolite is a non-ribosomally synthesized peptide.

In some instances, an analog of a substance (e.g., secondary metabolite) that shares one or more particular structural features, elements, components, or moieties with a reference substance. Typically, an analog shows significant structural similarity with the reference substance, for example sharing a core or consensus structure, but also differs in certain discrete ways. In some instances, an analog is a substance that can be generated from the reference substance, e.g., by chemical manipulation of the reference substance. In some instances, an analog is a substance that can be generated through performance of a synthetic process substantially similar to (e.g., sharing a plurality of steps with) one that generates the reference substance. In some instances, an analog is or can be generated through performance of a synthetic process different from that used to generate the reference substance. In some instances, an analog of a substance is the substance being substituted at one or more of its substitutable positions.

In some instances, an analog of a product comprises the structural core of a product. In some instances, a biosynthetic product is cyclic, e.g., monocyclic, bicyclic, or polycyclic, and the structural core of the product is or comprises the monocyclic, bicyclic, or polycyclic ring system.

In some instances, the structural core of the product comprises one ring of the bicyclic or polycyclic ring system of the product. In some instances, a product is or comprises a polypeptide, and a structural core is the backbone of the polypeptide. In some instances, a product is or comprises a polyketide, and a structural core is the backbone of the polyketide. In some instances, an analog is a substituted biosynthetic product comprising one or more suitable substituents.

### Systems for Biosynthetic Gene Cluster (BGC) Discovery

Also disclosed herein are systems designed to implement any of the disclosed machine learning-based methods for identifying BGCs in a genome. The systems may comprise, for example, one or more processors, and a memory unit communicatively coupled to the one or more processors and configured to store instructions that, when executed by the one or more processors, cause the system to: receive a first representation of at least one first genome as input; process the representation of the at least one first genome using a trained machine learning model configured to detect patterns of predicted protein domains encoded by genes belonging to a biosynthetic gene cluster (BGC); and output, based on detection of a pattern of predicted protein domains corresponding to a BGC in the first representation of the at least one first genome, a second representation of the at least one first genome that identifies a set of genes that belong to the BGC.

### Computing Devices and systemS

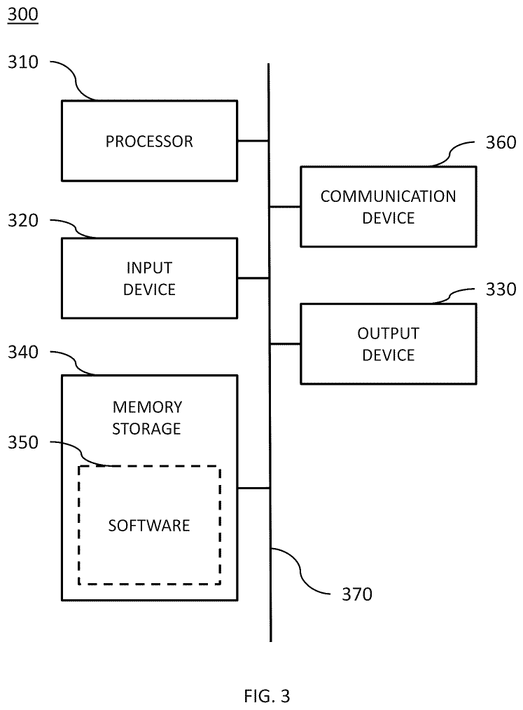

FIG. 3 illustrates an example of a computing device in accordance with one or more examples of the disclosure. Device 300 can be a host computer connected to a network. Device 200 can be a client computer or a server. As shown in FIG. 3, device 300 can be any suitable type of microprocessor-based device, such as a personal computer, workstation, server, or handheld computing device (portable electronic device), such as a phone or tablet. The device can include, for example, one or more of processor 310, input device 320, output device 330, storage 340, and communication device 360. Input device 320 and output device 330 can generally correspond to those described above, and they can either be connectable or integrated with the computer.

Input device 320 can be any suitable device that provides input, such as a touch screen, keyboard or keypad, mouse, or voice-recognition device. Output device 330 can be any suitable device that provides output, such as a touch screen, haptics device, or speaker.

Storage 340 can be any suitable device that provides storage, such as an electrical, magnetic, or optical memory including a RAM, cache, hard drive, or removable storage disk. Communication device 360 can include any suitable device capable of transmitting and receiving signals over a network, such as a network interface chip or device. The components of the computer can be connected in any suitable manner, such as via a physical bus 370 or wirelessly.

Software 350, which can be stored in memory/storage 340 and executed by processor 310, can include, for example, the programming that embodies the functionality of the present disclosure (e.g., as embodied in the devices described above).

Software 350 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 340, that can contain or store programming for use by or in connection with an instruction execution system, apparatus, or device.

Software 350 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Device 300 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, Tl or T3 lines, cable networks, DSL, or telephone lines.

Device 300 can implement any operating system suitable for operating on the network. Software 350 can be written in any suitable programming language, such as C, C++, Java, or Python. In various embodiments, application software embodying the functionality of the present disclosure can be deployed in different configurations, such as in a client/server arrangement or through a web browser as a web-based application or web service, for example.

### EXEMPLARY EMBODIMENTS

Among the provided embodiments are:
1. A computer-implemented method for identifying biosynthetic gene clusters comprising: receiving a first representation of at least one first genome as input;
   processing the representation of the at least one first genome using a trained machine learning model configured to detect patterns of predicted protein domains encoded by genes belonging to a biosynthetic gene cluster (BGC); and
   outputting, based on detection of a pattern of predicted protein domains corresponding to a BGC in the first representation of the at least one first genome, a second representation of the at least one first genome that identifies a set of genes that belong to the BGC.
2. The computer-implemented method of embodiment 1, wherein the first representation of the at least one first genome comprises a nucleotide sequence for the at least one first genome.
3. The computer-implemented method of embodiment 1 or embodiment 2, wherein the first representation of the at least one first genome comprises a vector representation of the at least one first genome, or an embedding thereof.
4. The computer-implemented method of any one of embodiments 1 to 3, wherein the first representation of the at least one first genome comprises a sequence of CDD, Gene3D, PANTHER, Pfam, ProSitePatterns, ProSiteProfiles, SUPERFAMILY, SMART, TIGRFAM, SFLD, Hamap, Coils, PRINTS, PIRSR, AntiFam, MobiDBLite, or PIRSF representations, or any combination thereof, of protein domains encoded by genes within the at least one first genome.
5. The computer-implemented method of embodiment 4, wherein the sequence of protein domain representations is generated by a process comprising retrieving a protein sequence for each gene in the at least one first genome and identifying protein domains in the protein sequence by sequence alignment against a database of CDD, Gene3D, PANTHER, Pfam, ProSitePatterns, ProSiteProfiles, SUPERFAMILY, SMART, or TIGRFAM, SFLD, Hamap, Coils, PRINTS, PIRSR, AntiFam, MobiDBLite, or PIRSF representations, respectively.
6. The computer-implemented method of embodiment 5, wherein the first representation of the at least one first genome further comprises associated gene ontology (GO) terms, an identification of any resistance genes present, an identification of additional regulatory elements, or an identification of additional epigenetic elements.
7. The computer-implemented method of any one of embodiments 4 to 6, further comprising encoding each protein domain representation in the sequence of CDD, Gene3D, PANTHER, Pfam, ProSitePatterns, ProSiteProfiles, SUPERFAMILY, SMART, or TIGRFAM, SFLD, Hamap, Coils, PRINTS, PIRSR, AntiFam, MobiDBLite, or PIRSF representations of protein domains as a vector representation of the at least one first genome using a representation learning system.
8. The computer-implemented method of embodiment 7, wherein the representation learning system comprises a graphical learning model, a deep autoencoder, Pfam2vec, word2vec, GloVe, or fastText.
9. The computer-implemented method of any one of embodiments 1 to 3, wherein the first representation of the at least one first genome comprises a sequence of annotations of genes within the at least one first genome based on a gene function and pathway mapping database.
10. The computer-implemented method of embodiment 9, wherein the pathway mapping database is KEGG.
11. The computer-implemented method of any one of embodiments 1 to 3, wherein the first representation of the at least one first genome comprises a sequence of annotations of genes within the at least one first genome based on a database comprising data for clusters of orthologous groups (COGs).
12. The computer-implemented method of embodiment 11, wherein the database comprising data for clusters of orthologous groups (COGs) is EggNOG.
13. The computer-implemented method of any one of embodiments 9 to 12, further comprising encoding the sequence of gene annotations as a vector representation of the at least one first genome using a representation learning system.
14. The computer-implemented method of embodiment 13, wherein the representation learning system comprises a graphical learning model, a deep autoencoder, Pfam2vec, word2vec, GloVe, or fastText.
15. The computer-implemented method of any one of embodiments 1 to 14, wherein the trained machine learning model comprises a deep learning model.
16. The computer-implemented method of embodiment 15, wherein the deep learning model comprises a supervised learning model or an unsupervised learning model.
17. The computer-implemented method of embodiment 15 or embodiment 16, wherein the deep learning model comprises a convolutional neural network, a long short-term memory network, or a transformer model.
18. The computer-implemented method of embodiment 15 or embodiment 16, wherein the deep learning model comprises a combination of components from a neural network, a convolutional neural network, a long short-term memory network, or a transformer neural network.
19. The computer-implemented method of any one of embodiments 1 to 18, wherein the machine learning model is trained using a training data set comprising data for a plurality of training genomes.
20. The computer-implemented method of embodiment 19, wherein the plurality of training genomes comprises a plurality of synthetic training genomes.
21. The computer-implemented method of embodiment 20, wherein one or more synthetic training genomes of the plurality of synthetic training genomes each comprise a set of gene sequences from an actual BGC randomly inserted into a BGC negative genome.
22. The computer-implemented method of embodiment 20 or embodiment 21, wherein one or more synthetic training genomes of the plurality of synthetic training genomes each comprise a set of gene sequences from a combination of actual positive BGC examples and synthetic negative BGC examples.
23. The computer-implemented method of any one of embodiments 1 to 22, wherein the second representation of the at least one first genome comprises a vector representation, a graph representation, or a tensor representation of the at least one first genome.
24. The computer-implemented method of any one of embodiments 1 to 23, further comprising evaluating a gene identified as belonging to the BGC to determine if it is a resistance gene.
25. The computer-implemented method of embodiment 24, wherein the resistance gene is an embedded target gene (ETaG) or a non-embedded target gene (NETaG).
26. The computer-implemented method of embodiment 24 or embodiment 25, further comprising performing an in vitro assay to test a secondary metabolite produced by the BGC in the at least first genome to which the resistance gene has been identified as belonging for activity against a resistance gene homolog, or protein encoded thereby, identified in a second genome that differs from the at least one first genome.
27. The computer-implemented method of any one of embodiments 24 to 26, further comprising performing an in vivo assay to test a secondary metabolite produced by the BGC in the at least first genome to which the resistance gene has been identified as belonging for activity against a resistance gene homolog, or protein encoded thereby, identified in a second genome that differs from the at least one first genome.
28. The computer-implemented method of embodiment 26 or embodiment 27, wherein the second genome comprises a mammalian genome, a human genome, an avian genome, a reptilian genome, an amphibian genome, a plant genome, a fungal genome, a bacterial genome, or a viral genome.
29. The computer-implemented method of any one of embodiments 1 to 28, wherein the at least one first genome comprises a eukaryotic genome or a prokaryotic genome.
30. The computer-implemented method of embodiment 29, wherein the at least one first genome is a eukaryotic genome, and the eukaryotic genome comprises a plant genome or a fungal genome.
31. The computer-implemented method of embodiment 29, wherein the at least one first genome is a prokaryotic genome, and the prokaryotic genome is a bacterial genome.
32. The computer-implemented method of any one of embodiments 1 to 31, wherein the first representation of the at least one first genome is input by a user of a system configured to perform the computer-implemented method.
33. A computer-implemented method comprising:
   receiving a sequence for at least one first genome as input;
   generating a first representation of the at least one first genome, wherein the first representation of the at least one first genome comprises a sequence of protein domain representations encoded by genes within the at least one first genome; and
   encoding each protein domain representation in the sequence of protein domain representations as a vector representation of the at least one first genome using a representation learning system.
34. The computer-implemented method of embodiment 33, wherein the sequence of protein domain representations encoded by genes within the at least one first genome comprises a sequence of CDD, Gene3D, PANTHER, Pfam, ProSitePatterns, ProSiteProfiles, SUPERFAMILY, SMART, TIGRFAM, SFLD, Hamap, Coils, PRINTS, PIRSR, AntiFam, MobiDBLite, or PIRSF representations, or any combination thereof, of protein domains encoded by genes within the at least one first genome.
35. The computer-implemented method of embodiment 34, wherein the sequence of protein domain representations is generated by a process comprising retrieving a protein sequence for each gene in the at least one first genome and identifying protein domains in the protein sequence by sequence alignment against a database of CDD, Gene3D, PANTHER, Pfam, ProSitePatterns, ProSiteProfiles, SUPERFAMILY, SMART, TIGRFAM, SFLD, Hamap, Coils, PRINTS, PIRSR, AntiFam, MobiDBLite, or PIRSF representations, respectively.
36. The computer-implemented method of embodiment 34 or embodiment 35, wherein the first representation of the at least one first genome further comprises associated gene ontology (GO) terms, an identification of any resistance genes present, an identification of additional regulatory elements, or an identification of additional epigenetic elements.
37. The computer-implemented method of any one of embodiments 33 to 36, wherein the representation learning system comprises a graphical learning model, a deep autoencoder, Pfam2vec, word2vec, GloVe, or fastText.
38. The computer-implemented method of any one of embodiments 33 to 37, wherein the representation learning system is trained on a corpus of annotated genomes, each comprising a sequence of CDD, Gene3D, PANTHER, Pfam, ProSitePatterns, ProSiteProfiles, SUPERFAMILY, SMART, TIGRFAM, SFLD, Hamap, Coils, PRINTS, PIRSR, AntiFam, MobiDBLite, or PIRSF representations, or any combination thereof, of protein domains encoded by genes within a genome of the corpus.
39. A system comprising:
   one or more processors; and
   a memory communicatively coupled to the one or more processors and configured to store instructions that, when executed by the one or more processors, cause the system to perform the method of any one of embodiments 1 to 38.
40. A non-transitory computer-readable storage medium storing one or more programs, the one or more programs comprising instructions which, when executed by one or more processors of a system, cause the system to perform the method of any one of embodiments 1 to 38.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A computer-implemented method for identifying biosynthetic gene clusters comprising:
receiving a first representation of at least one first genome as input;
processing the representation of the at least one first genome using a trained machine learning model configured to detect patterns of predicted protein domains encoded by genes belonging to a biosynthetic gene cluster (BGC); and
outputting, based on detection of a pattern of predicted protein domains corresponding to a BGC in the first representation of the at least one first genome, a second representation of the at least one first genome that identifies a set of genes that belong to the BGC.

2. The computer-implemented method of claim 1, wherein the first representation of the at least one first genome comprises a nucleotide sequence for the at least one first genome.

3. The computer-implemented method of claim 1, wherein the first representation of the at least one first genome comprises a vector representation of the at least one first genome, or an embedding thereof.

4. The computer-implemented method of claim 1, wherein the first representation of the at least one first genome comprises a sequence of CDD, Gene3D, PANTHER, Pfam, ProSitePatterns, ProSiteProfiles, SUPERFAMILY, SMART, TIGRFAM, SFLD, Hamap, Coils, PRINTS, PIRSR, AntiFam, MobiDBLite, or PIRSF representations, or any combination thereof, of protein domains encoded by genes within the at least one first genome.

5. The computer-implemented method of claim 4, wherein the sequence of protein domain representations is generated by a process comprising retrieving a protein sequence for each gene in the at least one first genome and identifying protein domains in the protein sequence by sequence alignment against a database of CDD, Gene3D, PANTHER, Pfam, ProSitePatterns, ProSiteProfiles, SUPERFAMILY, SMART, TIGRFAM, SFLD, Hamap, Coils, PRINTS, PIRSR, AntiFam, MobiDBLite, or PIRSF representations, respectively.

6. The computer-implemented method of claim 5, wherein the first representation of the at least one first genome further comprises associated gene ontology (GO) terms, an identification of any resistance genes present, an identification of additional regulatory elements, or an identification of additional epigenetic elements.

7. The computer-implemented method of claim 4, further comprising encoding each protein domain representation in the sequence of CDD, Gene3D, PANTHER, Pfam, ProSitePatterns, ProSiteProfiles, SUPERFAMILY, SMART, TIGRFAM, SFLD, Hamap, Coils, PRINTS, PIRSR, AntiFam, MobiDBLite, or PIRSF representations of protein domains as a vector representation of the at least one first genome using a representation learning system.

8. The computer-implemented method of claim 7, wherein the representation learning system comprises a graphical learning model, a deep autoencoder, Pfam2vec, word2vec, GloVe, or fastText.

9. The computer-implemented method of claim 1, wherein the first representation of the at least one first genome comprises a sequence of annotations of genes within the at least one first genome based on a gene function and pathway mapping database.

10. The computer-implemented method of claim 9, wherein the pathway mapping database is KEGG.

11. The computer-implemented method of 1, wherein the first representation of the at least one first genome comprises a sequence of annotations of genes within the at least one first genome based on a database comprising data for clusters of orthologous groups (COGs).

12. The computer-implemented method of claim 11, wherein the database comprising data for clusters of orthologous groups (COGs) is EggNOG.

13. The computer-implemented method of claim 9, further comprising encoding the sequence of gene annotations as a vector representation of the at least one first genome using a representation learning system.

14. The computer-implemented method of claim 13, wherein the representation learning system comprises a graphical learning model, a deep autoencoder, Pfam2vec, word2vec, GloVe, or fastText.

15. The computer-implemented method of claim 1, wherein the trained machine learning model comprises a deep learning model.

16. The computer-implemented method of claim 15, wherein the deep learning model comprises a supervised learning model or an unsupervised learning model.

17. The computer-implemented method of claim 15, wherein the deep learning model comprises a convolutional neural network, a long short-term memory network, or a transformer model.

18. The computer-implemented method of claim 15, wherein the deep learning model comprises a combination of components from a neural network, a convolutional neural network, a long short-term memory network, or a transformer neural network.

19. The computer-implemented method of claim 1, wherein the machine learning model is trained using a training data set comprising data for a plurality of training genomes.

20. The computer-implemented method of claim 19, wherein the plurality of training genomes comprises a plurality of synthetic training genomes.

21. The computer-implemented method of claim 20, wherein one or more synthetic training genomes of the plurality of synthetic training genomes each comprise a set of gene sequences from an actual BGC randomly inserted into a BGC negative genome.

22. The computer-implemented method of claim 20, wherein one or more synthetic training genomes of the plurality of synthetic training genomes each comprise a set of gene sequences from a combination of actual positive BGC examples and synthetic negative BGC examples.

23. The computer-implemented method of claim 1, wherein the second representation of the at least one first genome comprises a vector representation, a graph representation, or a tensor representation of the at least one first genome.

24. The computer-implemented method of claim 1, further comprising evaluating a gene identified as belonging to the BGC to determine if it is a resistance gene.

25. The computer-implemented method of claim 24, wherein the resistance gene is an embedded target gene (ETaG) or a non-embedded target gene (NETaG).

26. The computer-implemented method of claim 24 or claim 25, further comprising performing an in vitro assay to test a secondary metabolite produced by the BGC in the at least first genome to which the resistance gene has been identified as belonging for activity against a resistance gene homolog, or protein encoded thereby, identified in a second genome that differs from the at least one first genome.

27. The computer-implemented method of claim 24, further comprising performing an in vivo assay to test a secondary metabolite produced by the BGC in the at least first genome to which the resistance gene has been identified as belonging for activity against a resistance gene homolog, or protein encoded thereby, identified in a second genome that differs from the at least one first genome.

28. The computer-implemented method of claim 26, wherein the second genome comprises a mammalian genome, a human genome, an avian genome, a reptilian genome, an amphibian genome, a plant genome, a fungal genome, a bacterial genome, or a viral genome.

29. The computer-implemented method of claim 1, wherein the at least one first genome comprises a eukaryotic genome or a prokaryotic genome.

30. The computer-implemented method of claim 29, wherein the at least one first genome is a eukaryotic genome, and the eukaryotic genome comprises a plant genome or a fungal genome.

31. The computer-implemented method of claim 29, wherein the at least one first genome is a prokaryotic genome, and the prokaryotic genome is a bacterial genome.

32. The computer-implemented method of claim 1, wherein the first representation of the at least one first genome is input by a user of a system configured to perform the computer-implemented method.

33. A computer-implemented method comprising:
receiving a sequence for at least one first genome as input;
generating a first representation of the at least one first genome, wherein the first representation of the at least one first genome comprises a sequence of protein domain representations encoded by genes within the at least one first genome; and
encoding each protein domain representation in the sequence of protein domain representations as a vector representation of the at least one first genome using a representation learning system.

34-38. (canceled)

39. A system comprising:
one or more processors; and
a memory communicatively coupled to the one or more processors and configured to store instructions that, when executed by the one or more processors, cause the system to perform the method of claim 1.

40. A non-transitory computer-readable storage medium storing one or more programs, the one or more programs comprising instructions which, when executed by one or more processors of a system, cause the system to perform the method of claim 1.
